(19) United States
(12) Patent Application Publication
(10) Pub. No.: US 2021/0221890 A1
(43) Pub. Date: Jul. 22, 2021

(54) COMBINATION OF BETA-ADRENERGIC RECEPTOR ANTAGONISTS AND CHECK POINT INHIBITORS FOR IMPROVED EFFICACY AGAINST CANCER

(71) Applicant: Health Research, Inc., Buffalo, NY (US)

(72) Inventors: Elizabeth REPASKY, Buffalo, NY (US); Bonnie HYLANDER, Buffalo, NY (US)

(21) Appl. No.: 17/164,787

(22) Filed: Feb. 1, 2021

Related U.S. Application Data
(63) Continuation-in-part of application No. 15/557,211, filed on Sep. 11, 2017.
(60) Provisional application No. 62/132,286, filed on Mar. 12, 2015.

(51) Int. Cl. C07K 16/28; A61K 31/138; A61K 9/00; A61P 35/04

(52) U.S. Cl. CPC C07K 16/2818; A61P 35/04; A61K 9/0019; A61K 31/138

(57) ABSTRACT

Provided are methods for prophylaxis and/or therapy of cancer that include administering to an individual in need thereof an effective amount of a β-blocker and an immune checkpoint inhibitor such that growth of cancer in the individual is inhibited. Patients include those diagnosed with or at risk for a wide variety of cancer types. Methods are provided for cancer treatment in individuals who are resistant to checkpoint inhibitor monotherapies. Greater than additive anti-cancer effects may be achieved.

### CROSS REFERENCE TO RELATED APPLICATIONS

This application is a continuation-in-part of U.S. patent application Ser. No. 15/557,211, filed Sep. 11, 2017, now pending, which is a National Phase of International Application No. PCT/US2016/022277, filed Mar. 14, 2016, which claims priority to U.S. provisional patent application No. 62/132,286, filed Mar. 12, 2015, the disclosures of each of which are incorporated herein by reference.

### FIELD

The present disclosure relates generally to anti-cancer approaches and more specifically to combinations of β-adrenergic receptor antagonists and checkpoint inhibitors.

### BACKGROUND

There is much evidence that patients are able to mount an immune response against their own tumors, however tumors often remain unharmed because of their ability to actively suppress, or even kill, immune cells. There are many approaches being developed to overcome this suppression and develop effective immunotherapies for cancer patients. Among the most promising are vaccines and adoptive T-cell transfer. However, these therapies are also vulnerable to tumor mechanisms of immunosuppression. Recently, it has been found that tumors employ strategies which mimic the natural mechanisms by which the immune response is down-regulated, specifically by expression of so-called immune checkpoint ligands which interact with receptors on immune cells to block their activity. “Immune checkpoint inhibitors” have been developed to block these interactions. One such example of these inhibitors are antibodies to PD-1 (programmed cell death-1), a receptor on the T-cells which when bound by PD-L1 (a molecule often expressed on tumor cells) inhibits the activity of the T-cells and suppresses the anti-tumor immune response. However, there is an ongoing need for compositions and methods that can enhance the efficacy of check point inhibitors. The present disclosure relates to this need.

### SUMMARY

The present disclosure relates generally to methods for prophylaxis and/or therapy of cancer. The method comprises administering to an individual in need thereof an effective amount of a β-adrenergic receptor antagonist (β-blocker) and an immune checkpoint inhibitor such that growth of cancer in the individual is inhibited.

In certain approaches the individual is diagnosed as, or is suspected of having, or develops a cancer that is resistant to an immune checkpoint inhibitor. In certain implementations the individual is not being treated, and/or has not previously been treated with a β-blocker. In certain approaches methods of this disclosure comprise selecting an individual who has a cancer that exhibits resistance to treatment with a checkpoint inhibitor, wherein the individual is not treated with a β-blocker when the resistance is exhibited, and administering to the individual the checkpoint inhibitor and the β-blocker such that growth of the cancer is inhibited.

Methods of the disclosure can be performed using any suitable checkpoint inhibitors and β-blockers, and can include using more than one of either of these types of agents. In particular and non-limiting examples the immune checkpoint inhibitor is selected from anti-programmed cell death protein 1 (PD-1) antibody or PD-1 binding fragment thereof, or an anti-PD-1 principal ligand (PD-L1) antibody or PD-L1 binding fragment thereof. PD-1 and PD-L1 are well characterized in the art.

In certain implementations the β-blocker comprises a nonselective β-blocker and thus is pertinent to the three presently known types of beta receptors (β1, β2 and β3 receptors). In certain examples the β-blocker interferes with or more of these receptors binding to their endogenous ligands and thus may be competitive antagonists for any β-adrenergic receptor(s).

In certain approaches administering the β-blocker and the immune checkpoint inhibitor results in a greater than additive inhibition of growth of the cancer.

In certain approaches the individual treated with a combination of this disclosure has a cancer that was treated with the checkpoint inhibitor without the β-blocker, wherein the cancer was resistant to the checkpoint inhibitor, and wherein the β-blocker and the immune checkpoint inhibitor are subsequently administered to the individual such that growth of the cancer is inhibited.

In one aspect the disclosure provides a method comprising selecting an individual who has a cancer that exhibits resistance to treatment with a checkpoint inhibitor, wherein the individual is not treated with a β-blocker when the resistance is exhibited, and administering to the individual the checkpoint inhibitor and the β-blocker such that growth of the cancer is inhibited.

### BRIEF DESCRIPTION OF FIGURES

FIG. 1. Graph of data obtained using Pan02 pancreatic cell line tumors in C57BL/6 mice. Treatment of the mice with the β-blocker propranolol significantly improved the efficacy of anti-PD-1 checkpoint inhibition. *p<0.05.

FIG. 2. Graph of data demonstrating that β-AR blockade improves the efficacy of anti-PD-1 immunotherapy in a breast cancer model (4T1 cell line in BALB/c mice). Data depict absolute tumor volume. Neither propranolol nor anti-PD-1 mAb alone has any impact on tumor growth. But a combination of β-blocker and checkpoint inhibitor results in a greater than additive reduction in absolute tumor volume growth.

FIG. 3. Graph of data from experiment depicted in FIG. 2 graphed as changes in relative tumor volume, demonstrating that β-AR blocker improves the efficacy of anti-PD-1 immunotherapy in a breast cancer model.

FIGS. 4A and 4B represent data obtained in a melanoma model. Individual tumor growth curves (FIG. 4A) and tumor growth rates (FIG. 44B) show a combination of anti-PD-1 and β-blocker is effective in melanoma tumors that do not respond to anti-PD-1 as a monotherapy.

FIG. 5 shows a spider plot for tumor response for all nine patients that took part in a Phase I study (P1-P9). Spider plot of the change in the sum of tumor diameters over time for the nine evaluable patients on study. 0 on the X-axis corresponds to the time of the baseline scan and percentage change in tumor size from baseline is shown on the Y-axis. P1, P2 and P3=10 mg propranolol; P4, P5 and P6=20 mg propranolol; P7, P8 and P9=30 mg propranolol.

### DESCRIPTION

The disclosure relates to methods for cancer therapy comprising administering to an individual in need a combination of an immune checkpoint inhibitor and one or more β-blockers.

In embodiments, the individual in need of treatment in accordance with this disclosure is any mammal, including but not limited to a human. The cancer type is not particularly limited, other than being a cancer type for which immune checkpoint inhibition may be a suitable prophylactic and/or therapeutic approach. In embodiments, the individual is at risk for, is suspected of having, or has been diagnosed with a cancer. In embodiments, the cancer is lung, colon, breast, pancreatic, brain, liver, bladder, kidney, melanoma, ovary, testicular, esophageal, gastric, fibrosarcoma, rhabdomyosarcoma, head and neck, renal cell, thyroid, or a blood cancer.

The disclosure is also pertinent to approaching cancers that are or may become resistant to treatment with one or more immune checkpoint inhibitors. Thus in certain implementations the individual has been previously treated for cancer with a checkpoint inhibitor, but was not given any β-blockers while being treated with the checkpoint inhibitor, and the cancer was initially resistant, or develops resistance, to the checkpoint inhibitor treatment. The disclosure thus includes selecting an individual who has cancer that is resistant to a checkpoint inhibitor as a monotherapy, and administering to the individual a checkpoint inhibitor and a β-blocker. The individual who is resistant to a checkpoint inhibitor as a monotherapy accordingly means an individual who was administered a checkpoint inhibitor for the cancer, but was not also administered a β-blocker, and the cancer was resistant to the treatment that included the checkpoint inhibitor but not the β-blocker. The monotherapy may have included other anti-cancer agents or other interventions, so long as such other agents did not include the β-blocker that is subsequently used in a combination therapy of this disclosure. In one embodiment the individual who is treated with a combination approach described herein has never been previously treated with a β-blocker. In certain embodiments the individual who is treated with a combination therapy of this disclosure has not been diagnosed with, or is not suspected of having, or is not a risk for developing a non-cancerous condition for which a β-blocker would ordinarily be prescribed.

In certain embodiments a combination of an immune checkpoint inhibitor and a β-blocker exerts a synergistic effect against cancer, which may comprise but is not limited to a greater than additive inhibition of cancer progression, and/or a greater than additive inhibition of an increase in tumor volume, and/or a reduction in tumor volume, and/or a reduction in tumor growth rate, and/or an eradication of a tumor and/or cancer cells. The method may also result in a prolonging of the survival of the individual.

The disclosure also comprises monitoring the treatment of an individual who is receiving a combination of an immune checkpoint inhibitor and a β-blocker. This approach comprises administering the combination of an immune checkpoint inhibitor and a β-blocker as a cancer treatment, testing the individual and/or a biological sample from the individual to determine the efficacy of the combination therapy, and if determined to be necessary, adjusting the combination therapy by, for example, changing the amount of the immune checkpoint inhibitor or the β-blocker, or both, and/or changing the type of immune checkpoint inhibitor and or the β-blocker. Retesting and changing the combination therapy may also be performed.

As is known in the art, β-blockers comprise a class of drug compounds that are typically used for management of cardiac arrhythmias, inhibition of secondary myocardial infarction, management of hypertension, and other indications. The present disclosure includes using any one or any combination of β-blockers that are selective or non-selective antagonists of any one or any combination of the three presently known types of beta receptors (β1, β2 and β3 receptors), or otherwise interfere with one or more of these receptors binding to their endogenous ligands, i.e., epinephrine and/or other stress hormones. Thus, in embodiments, β-blockers used in this disclosure comprises a class of competitive antagonists for β-adrenergic receptors. In one embodiment, the β-blocker is a nonselective β-blocker, such as a sympatholytic β-blocker. In one embodiment, the β-blocker is propranolol. In certain embodiments the β-blocker is selected from Bucindolol, Carteolol, Carvedilol, Labetalol, Nadolol, Oxprenolol, Penbutolol, Pindolol, Sotalol, and Timolol.

The immune checkpoint inhibitor used in combination with the one or more β-blockers can be any immune checkpoint inhibitor. As is known in the art, an example of an immune checkpoint is the transmembrane programmed cell death 1 protein (PDCD1, PD-1; also known as CD279) and its ligand, PD-1 ligand 1 (PD-L1, CD274). In normal, non-malignant physiology, PD-L1 on the surface of a cell binds to PD1 on the surface of an immune cell, which inhibits the activity of the immune cell. PD-L1 up-regulation on cancer cell surfaces is thought to facilitate evasion of the host immune system, at least in part by inhibiting T cells that would otherwise target the tumor cell. In alternative embodiments, other immune checkpoints can be inhibited, such CTLA-4.

In embodiments, any one or more checkpoint inhibitors can be combined with any one or more β-blockers for use in the methods of this disclosure. In certain embodiments, the checkpoint inhibitors that are combined with the β-blockers comprise antibodies that bind to PD-1, or anti-PD-L1, such as Nivolumab. An example of a PD-1 directed antibody is pembrolizumab.

In another embodiment, the checkpoint inhibitor is an antibody that targets CTLA-4, such as Ipilimumab. In another embodiment the checkpoint inhibitor is targets CD366 (Tim-3), which is a transmembrane protein also known as T cell immunoglobulin and mucin domain containing protein-3.

In alternative embodiments, the checkpoint inhibitors comprise small molecules or other agents that disrupt the immune checkpoint that is exploited by cancer cells to evade cell-mediated or other immune-mediated targeting.

Those skilled in the art, given the benefit of the present disclosure, will recognize how to determine an effective amount of the combination of checkpoint inhibitor and β-blocker for treatment of cancer. In general, and without intending to be bound by any particular theory, it is expected that the amounts of each of these agents that are used and/or tested currently in humans for their separate indications will also be effective in the presently provided combination approach. But modifications can be made by medical professionals based on known conditions, such as the size, age, gender and overall health profile of the individual, the type and stage of the cancer, and other conditions and risk factors that will be otherwise apparent to those skilled in the art.

In embodiments, the β-blocker comprises propranolol. In embodiments, the propranolol is administered in an amount of at least 10 mg. In embodiments, the propranolol is administered in an amount of 10 mg-50 mg, inclusive, and including all numbers and ranges of numbers there between. In embodiments, the propranolol is administered in an amount that is 10 mg, 20 mg, or 30 mg. Any described dosage can be at least one time per day. In an embodiment, the described dosage of the propranolol is administered at least two times a day, or only two times per day (BID). The described propranolol can be combined with any suitable checkpoint inhibitor dosing regimen. In embodiments, the described propranolol is administered with pembrolizumab (formerly lambrolizumab). In embodiments, the pembrolizumab is administered intravenously (i.v.). In embodiments, the pembrolizumab is administered daily, weekly, bi-weekly, or one every three weeks. In an embodiment, pembrolizumab is administered once every three weeks in a suitable amount. In an embodiment, a suitable amount of pembrolizumab comprises 100-500 mg, inclusive, and including all ranges of numbers there between. In an embodiment, a suitable amount of pembrolizumab comprises 200 mg. In an embodiment, a suitable dosing regimen comprises i.v. administration of pembrolizumab in an amount of 200 mg once every three weeks. In an embodiment, the disclosure provides for administration of from 10-30 mg propranolol BID and pembrolizumab in an amount of 200 mg once every three weeks. In an embodiment, the described dosing is administered to an individual with melanoma. In an embodiment, the effect of combined administration of the propranolol and pembrolizumab is greater than the effect of administering pembrolizumab alone, or administering propranolol alone. In an embodiment, the disclosure provides for administration of 30 mg of propranolol BID and 200 mg pembrolizumab by i.v. once every three weeks to a human individual diagnosed with melanoma.

Thus, in embodiments, administering the checkpoint inhibitor and the β-blocker has a greater than additive effect on tumor inhibition, relative to use of either agent alone. A greater than additive effect can be determined by comparing the effects of one or both of the agents to any suitable reference, including but not limited to a predetermined value. In embodiments, a described dosing inhibits the growth of a tumor, e.g., inhibits an increase in tumor volume, or causes a decrease in tumor volume during the course of the treatment. In embodiments, the described approach reduces the volume of a melanoma tumor at least during the course of treatment.

In embodiments, one or more β-blockers and one or more immune checkpoint inhibitors are administered concurrently. In embodiments, the one or more β-blockers and one or more immune checkpoint inhibitors are combined into a single pharmaceutical formulation. In embodiments, the one or more β-blockers and the one or more immune checkpoint inhibitors are administered sequentially. The β-blocker and immune checkpoint inhibitor can be administered via any suitable route, including but not necessarily limited to intravenous, intramuscular, subcutaneous, oral, and parenteral routes.

In an embodiment, the combination therapy has a greater than additive inhibition of tumor growth, which may be determined using any suitable measurement, non-limiting examples of which include determining tumor volume or tumor growth rate. The combination therapy can be combined with any other, conventional cancer therapies, including but not limited to surgical and chemotherapeutic approaches.

The following specific examples are provided to illustrate the invention, but are not intended to be limiting in any way.

### Example 1

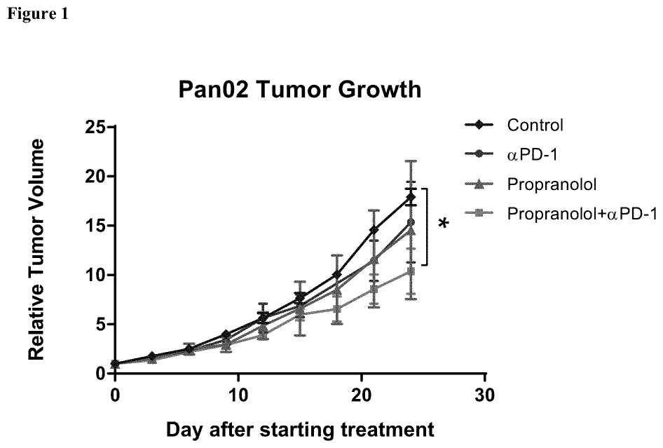

This Example shows that a combination of a β-blocker and an immune checkpoint inhibitor inhibits tumor growth, and demonstrates that the combination is capable of eliciting a greater than additive inhibition of tumor growth. In particular, and without intending to be constrained by any particular, theory, it is considered that combining specific blockade of a stress response with a β-blocker will reverse the systemic immunosuppression caused by stress, such as cancer, and when given in combination with a immune checkpoint inhibitor, will result in both improved activation (i.e., effect of the β-blocker) and sustained activity (i.e., effect of the checkpoint inhibitor) of immune cells and result in significantly improved efficacy of immune cells against the tumor. Accordingly, as shown in FIG. 1, β-AR blockade improves the efficacy of anti-PD-1 immunotherapy in a pancreas cancer model. To obtain the data presented in FIG. 1, a murine pancreatic tumor (Pan02) was engrafted in C57BL/6 mice. When tumors reached an average size of 50-100 mm³, tumor bearing mice were assigned to one of four experimental groups and treatment was initiated. Groups of mice received: (1) saline (control), (2) anti-PD-1 antibody (BIO X CELL®, 200 μg/dose every 3 days), (3) the β-adrenergic signaling antagonist propranolol (10 mg/kg daily by intraperitoneal injection) or (4) combination therapy. Tumor growth was monitored and graphed as relative tumor volume compared to each tumor's starting volume. As can be seen in FIG. 1, neither anti-PD-1 nor propranolol alone caused a statistically significant decrease in tumor growth. However, tumor growth was statistically significantly slowed in mice receiving the combination therapy (Students t-test) such that the effect was greater than additive. The data accordingly indicate that the combination therapy can be effective against pancreatic tumors that are resistant to treatment with the checkpoint inhibitor alone.

### Example 2

This Example demonstrates that β-AR blockade improves the efficacy of anti-PD-1 immunotherapy in a breast cancer model. These data, and those obtained using a B16 tumor data provided below were obtained as described for FIG. 1, except β-AR blockade (the propranolol treatment) was started three days before administering the anti-PD-1 checkpoint inhibitor, and this 4T1 breast cancer model used BALB/c mice.

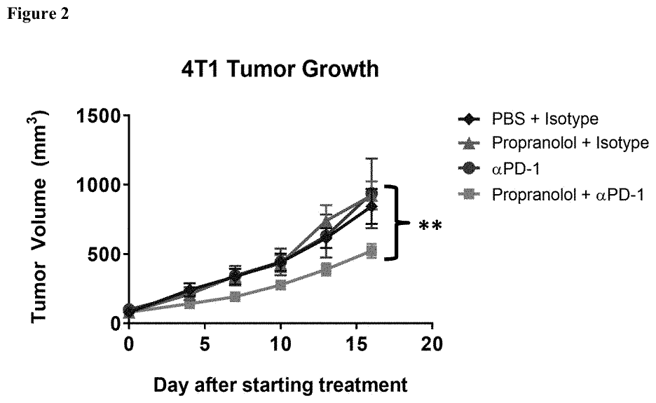

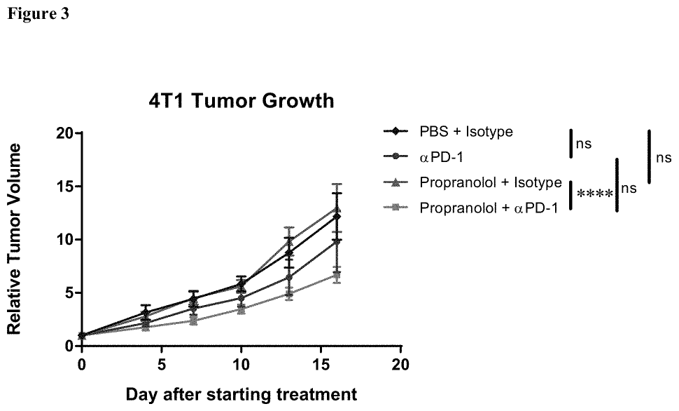

As can be seen from FIGS. 2 and 3 (measuring absolute tumor volume and relative tumor volume, respectively), neither propranolol nor anti-PD-1 mAb alone has any impact on tumor growth. But a combination of β-AR antagonist and checkpoint inhibitor results in a greater than additive reduction in absolute tumor volume growth. Thus, as with the pancreatic cancer model, the data demonstrate that this combination approach is effective even in breast cancer tumors that do not respond to anti-PD-1 as a monotherapy.

### Example 3

This Example demonstrates that β-AR blockade improves the efficacy of anti-PD-1 immunotherapy in a melanoma model.

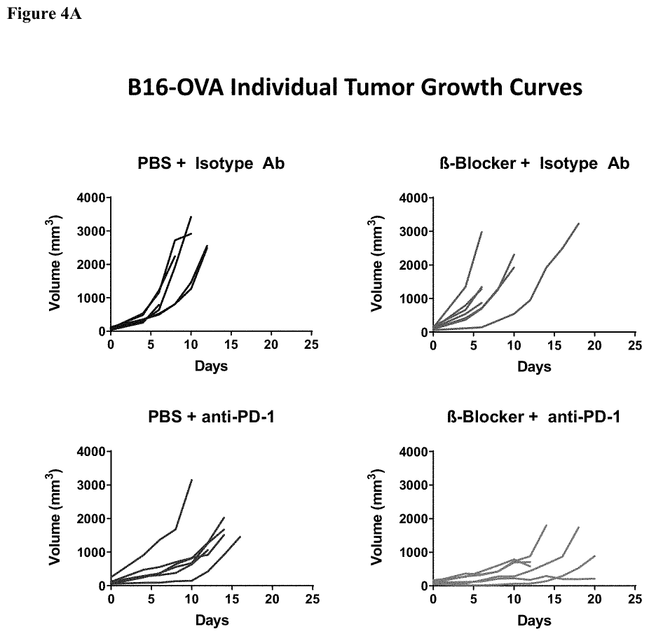

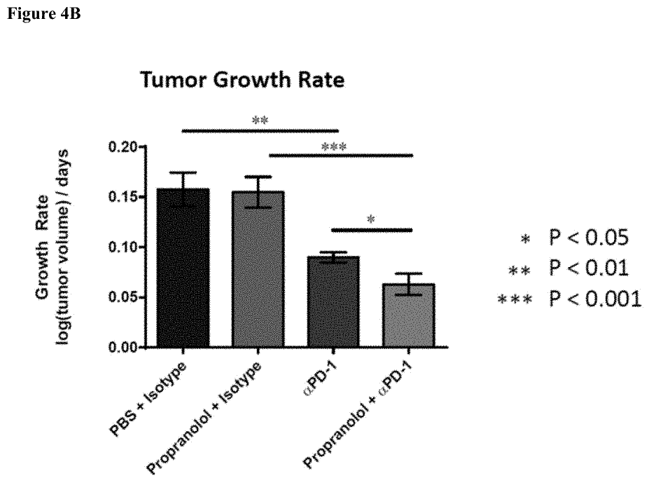

As can be seen from FIGS. 4A and 4B, which summarize data reflecting individual tumor growth curves (FIG. 4A) and tumor growth rates (FIG. 4B), the combination approach is effective even in melanoma tumors that do not respond to anti-PD-1 as a monotherapy.

While the invention has been described through specific embodiments, routine modifications will be apparent to those skilled in the art and such modifications are intended to be within the scope of the present invention. But as can be seen from the foregoing examples, and again without intending to be constrained by any particular theory, it is believed that the presently described combination approach is superior to the use of either single agent because the β-blocker will act to reverse the high levels of immunosuppressive cells (MDSC and T-regs) induced by the tumor, thus allowing the activation of the anti-tumor immune response, while the checkpoint inhibitor (shown here with an anti-PD-1 antibody) will bind to the PD-1 molecule (or other checkpoint molecule depending on the inhibitor), which is in this case expressed on the surface of activated immune cells (cytoxic T lymphocytes) and prevent ligation by tumor expressed PD-L1 which would otherwise lead to CTL inhibition. Therefore, this two-pronged approach both allows activation of the immune cells and sustains that activity long enough to support anti-tumor efficacy, even in distinct tumor types that are resistant to at least one checkpoint inhibitor.

### Example 4

This Example provides results from a phase I human clinical trial, combining an anti-PD-1 antibody (pembrolizumab) with the non-selective β-blocker, propranolol. To obtain the results presented in this Example, a 3+3 dose-escalation for propranolol twice a day (BID) with pembrolizumab (200 mg every 3 weeks) was performed. Specifically, metastatic melanoma patients received increasing doses of propranolol in cohorts of 10, 20 and 30 mg BID. No dose-limiting toxicities were observed. Objective response rate was 78%.

Methods

Study Population

Eligible patients were recruited from the melanoma clinic at Roswell Park Comprehensive Cancer Center (Roswell Park), Buffalo, N.Y. Eligibility criteria included adult patients (aged ≥18 years), Eastern Cooperative Oncology Group (ECOG) performance status of 0 (indicating no symptoms) or 1 (indicating mild symptoms) with treatment naïve, histologically confirmed unresectable stage III or IV melanoma, with good organ function, and measurable disease on computed tomography (CT; preferred) or magnetic resonance imaging scans per immune-modified Response Evaluation Criteria In Solid Tumors (imRECIST) guidelines and absence of symptomatic brain metastases. Key exclusion criteria were prior therapy with PD-1/PD-L1 inhibitors, chronic autoimmune disease or other immunodeficiency syndromes, contraindication to use of β-blockers (uncontrolled depression, grade III or IV heart failure, severe asthma or COPD, uncontrolled type 1 or type 2 diabetes mellitus with HbA1C >8.5 or fasting plasma glucose >160 mg/dl, symptomatic peripheral arterial disease or Raynaud's syndrome), and current or past use of β-blockers or calcium channel blockers in the last 2 years.

Design

This Example describes an open label, single arm, non-randomized, single center, phase Ib study. Dose-escalation followed a “3+3” design, and no intra-patient dose-escalation was allowed. Eligible patients were treated with standard of care pembrolizumab 200 mg every 3 weeks i.v. and progressively increasing propranolol dosing from 10 mg (dose level 1), 20 mg (dose level 2) to 30 mg (dose level 3) twice a day, until 2 years on study or disease progression or dose-limiting toxicities (DLT). A total of 9 patients were accrued. At the cutoff date, a total of 4 patients continue to be on the study treatment per protocol.

Objectives and Endpoints

An objective of this Example was to assess the safety and efficacy [ORR (overall response rate within 6 months of starting therapy)] of combination of pembrolizumab with increasing doses of propranolol for unresectable stage III and metastatic melanoma.

Safety outcomes were assessed by physical examination, laboratory findings, vital signs, and electrocardiogram. Adverse events (AE) were graded by Common Terminology Criteria for Adverse Events (CTCAE) v4.03. No AE due to propranolol doses of 10 mg-30 mg twice a day were anticipated. Nevertheless, a serious AE/DLT due to propranolol was defined as any life-threatening adverse event (e.g. symptomatic bradycardia or symptomatic hypotension) which would mandate recruitment per the 3+3 design. Otherwise a DLT was defined as grade 3 and higher immune-related adverse event (irAE) pneumonitis, colitis, hepatitis, nephritis, anemia, myositis, cardiomyositis, as defined by CTCAE v4.03, new onset diabetic ketoacidosis, Guillain-Barre syndrome or any other condition which the investigator believed to be an immune mediated adverse event and necessitated stopping therapy. Endocrinopathies were not included as DLTs, as the hormones will be replaced.

Objective response was defined as confirmed complete response (CR) or confirmed partial response (PR) among all treated patients with measurable disease at baseline.

Additional objectives were to analyze efficacy as progression-free survival (PFS) and OS. PFS was measured from treatment initiation to time of disease progression or death, while OS was measured from the date of starting treatment until date of death or censoring. Exploratory objectives included analysis of biomarkers over time on study.

Assessments

Patients were assessed for tumor response according to imRECIST every 12 weeks (+/−14 days) for the first 6 months, and then per physician discretion until confirmed disease progression or toxicities. Safety assessments occurred at each clinic visit.

Exploratory Analyses

Baseline tumor tissues, archival or fresh biopsy, were analyzed. Participants underwent serial blood collection into heparin and EDTA tubes for analysis of several biomarkers in peripheral blood. Patients completed the validated perceived stress scale (PSS) questionnaire Cohen, S., T. Kamarck, and R. Mermelstein, A global measure of perceived stress. J Health Soc Behav, 1983. 24(4): p. 385-96] at baseline and additional time points to measure and quantify patient reported stress level perception. The results are reported as low stress (scores 0-13), moderate stress (scores 14-26) and high stress (scores 27-40).

Tissue Collection and analyses: Participants underwent tumor tissue collection at baseline for diagnosis, prior to study enrollment. 2/9 patients had a fresh biopsy and archival tissue was used for 7/9 patients. Formalin fixed paraffin embedded tissues were sectioned at 4 μm for multispectral immunofluorescence staining with antibodies against the following markers: CD8 (Dako, clone CD8/144B, dil 1:250), CD4 (Dako, clone 4B12, ready to use), Foxp3 (Abcam, clone 236A/E7, dil 1:125), CD14 (Cell Marque, clone EPR3563, dil 1:100), CD15 (Dako, clone Carb-3, dil 1:50), PDL1 (Abcam, clone SP142, dil 1:100) and DAPI. Multispectral staining was performed after antibodies were optimized for standard immunohistochemistry and uniplex immunofluorescence staining. Slides were imaged using the Vectra Polaris spectral imaging system (PerkinElmer). Slides were initially scanned at ×4, visualized using Phenochart viewer (PerkinElmer) and five tumor areas per case were selected for scanning at high resolution (×20). Each fluorophore from PerkinElemer Opal™ kit was measured using a separate filter cube corresponding to its emission wavelength. The images were unmixed using a spectral library and individual fluorophores were separated with inForm™ software. The immune cell populations were quantified using cell segmentation and phenotype cell tool from inForm 1.1 (PerkinElmer). Threshold for positive staining and accuracy of phenotypes were confirmed by pathologist supervision (AKW). The individual markers from the panel were quantified and plotted as the average of the positive staining cells across the regions of interest.

Flow cytometry of peripheral blood: Flow cytometry was used to quantify MDSC and regulatory T cell (Treg) populations in freshly isolated peripheral blood samples from heparinized tubes. An eight-color panel comprised of CD11b FITC, CD16 PE, CD45 PerCP, CD33 PECy7, HLADR APC, CD14 APCH7, CD15 V450; a lineage dump consisting of CD3, CD19, and CD56 (all conjugated to BV510) was used to measure eMDSC, mMDSC, and gMDSC subsets (Panel 1). Separately, a six-color panel comprised of CD8 FITC, CD25 PE, CD4 PerCP, CD3 PECy7, CD45 APCH7, and CD127 BV421 was employed to measure T cell subsets (Panel 2). WinList software (version 8.0; Verity Software House) was employed for the analysis of flow cytometric data. Analyzed data were reported as absolute cell count (cells per μL), or separately as the percentage of CD45+ events. MDSC populations were quantified according to known phenotypic definitions. In brief, to quantify eMDSC, mononuclear cells (defined on the basis of their CD45 expression profile and light scatter characteristics) were sequentially gated to bivariate plots of HLADR vs. DUMP, CD14 vs. CD15, and CD33 vs. CD11b; where eMDSC were further defined as HLADR^{low/-}, DUMP−, CD14−, CD15−, CD33+, and CD11b+. To quantify mMDSC, mononuclear cells were sequentially gated to bivariate plots of CD11b vs. CD15 and HLADR vs. CD14; where mMDSC were further defined as CD11b+, CD15−, HLADR^{low/-}, and CD14+. To quantify gMDSC, mononuclear cells were sequentially gated to bivariate plots of CD14 vs. CD15 and CD11b vs. SSC-A; where gMDSC were further defined as CD14−, CD15+, and CD11b+. Separately, T cell subsets were quantified using Panel 2. Helper T cells were defined as CD3⁺, CD4⁺, CD8⁻ and cytotoxic T cells were defined as CD3⁺, CD4⁻, CD8⁺. To quantify Tregs, CD3⁺, CD4⁺, CD8⁻ T cells were gated to a bivariate plot of CD25 vs. CD127; where Tregs were further defined as CD25⁺, CD127(dim).

Chemokines/cytokines in peripheral blood: Plasma was collected from EDTA blood and stored as aliquots at −80° C. 29-plex MILLIPLEX® MAP Human Cytokine/Chemokine Magnetic Bead Panel 96-Well Plate Assay was used to examine blood plasma levels of cytokines and chemokines. Wash buffer, sheath fluid, serum matrix, samples and standards were prepared in accordance with manufacturer's protocol. The resultant data was analyzed using Upstate BeadView software for median fluorescence intensity (MFI) using a 5-parameter logistic curve-fitting method to calculate analyte concentrations in both samples and control wells.

Statistical Analysis

A standard 3+3 design was used, with 3 dose levels, and requiring up to n=18 subjects. In the primary analysis, adverse events and objective response are summarized by dose level using frequencies and relative frequencies.

For intra-dose analysis, peripheral blood biomarkers were summarized by dose-level and time-point using mean plots (+/−standard error). For intra-dose-level comparisons, the markers were modeled as a function of time-point and a random subject effect using a linear mixed model. An F-test about the main effect of time was used to evaluate whether marker expression changes over time. Additionally, the mean level at each time-point was compared to baseline using Dunnet adjusted tests. For inter-dose-level comparisons, percent change was calculated from baseline for each biomarker. The mean percent change was compared between dose levels using an ANOVA model, with pairwise comparisons made using a Tukey adjustment. All model assumptions were verified graphically, and transformations were applied as appropriate. All analyses were completed in SAS v9.4 (Cary, N.C.) at a significance level of 0.05.

Results

As of the data cut-off date, nine patients with cutaneous melanoma, treatment-naïve to PD-1/PD-L1 and CTLA-4 inhibitors have completed enrollment for the phase I safety study. The median age of patients on the study was 65 years (35-96). Six patients were female (67%), and all patients were Caucasian. At baseline, 6 patients had an ECOG performance score of 0 (67%), 5 patients had Mlc disease (56%) and 3 patients had elevated LDH (33%). The baseline PSS score ranged from 6-30, with a median score of 13. Four of 9 patients (44%) remain on study treatment. Primary reasons for study discontinuation were adverse events in 2 patients (22%) and disease progression in 3 patients (33%). Baseline patient and disease characteristics are summarized in Table 1.

TABLE 1

| Characteristics | Total Patients (N = 9) | |
|---|---|---|
| Median age at diagnosis (range), year | 65 | (35-76) |
| Males, N (%) | | |
| ECOG, N (%) | | |
| 0 | 6 | (67%) |
| 1 | 3 | (33%) |
| Elevated baseline LDH, N (%) | 3 | (33%) |
| Metastatic Stage, N (%) | M1b: 4 (44%) | |
| | M1c: 5 (56%) | |
| PD-L1 positive tumor (>1%), N (%) | 7/8 | (88%)* |

| | |
|---|---|
| Median baseline PSS score | 13 |

ECOG, Eastern Cooperative Oncology Group;
LDH, Lactate dehydrogenase;
PD-L1, Programmed cell death-Ligand-1;
PSS, Perceived Stress Scale
*PD-L1 could not be assessed in one patient.

Safety and Tolerability

Treatment-related adverse events (TRAEs) occurred in all 9 patients. All but 1 patient had TRAEs that were grade 2 or lower. The most commonly reported TRAEs were fatigue, rash and vitiligo which occurred in 4/9 (44%) patients.

Serious TRAEs leading to discontinuation of therapy occurred in 2 patients, both in the 20 mg BID cohort: hemophagocytic lymphohistiocytosis (HLH) and labyrinthitis. Two Grade ≥3 AEs were reported in 1 patient. That patient developed a grade 3 increase in alanine aminotransferases (ALT), which was treated with oral prednisone. Subsequently he was found to have hepatitis C for which he was treated successfully. Later, he had a hospital admission complicated by necrotizing fasciitis, deep vein thrombosis, and HLH. No dose-limiting toxicities (DLTs) were observed at any of the three dose levels.

Antitumor Activity

By the cutoff date, the median follow-up was 15.6 (range, 5.4-24.2) months. The median number of pembrolizumab cycles received were 7.5 (2-32). Objective responses were noted at all three dose levels (Table 2). Objective response rate was 7/9 (78%) on the study.

TABLE 2
Best responses per immune-modified RECIST and current
status of patients on study as of cutoff date.

| | | | | Time to | Time to | |
|---|---|---|---|---|---|---|
| Dose | Patient | Best | Follow up | best response | progression | |
| group | number | response | (in months) | (in months) | (in months) | Current status |
| 10 mg | P1 | Stable | 24.2 | 2.8 | 8.2 | Received radiation to |
| | | disease | | | | the enlarging lymph |
| | | | | | | node. Currently on |
| | | | | | | pembrolizumab and |
| | | | | | | propranolol off study |
| | P2 | Partial | 23.9 | 3.0 | 10.2 | Progressive disease, |
| | | response | | | | and currently being |
| | | | | | | treated with later lines |
| | | | | | | of therapy |
| | P3 | Partial | 23.1 | 2.8 | — | Continues treatment |
| | | response | | | | per protocol |
| 20 mg | P4 | Partial | 18.9 | 3.0 | — | Off study due to |
| | | response | | | | toxicity. Continues to |
| | | | | | | maintain response |
| | P5 | Partial | 15.5 | 5.8 | 11.6 | Off study due to |
| | | response | | | | toxicity. Patient had |
| | | | | | | metastatectomy for |
| | | | | | | progressive disease |
| | P6 | Progressive | 7.9 | — | 1.1 | Patient died |
| | | disease | | | | |
| 30 mg | P7 | Partial | 9.0 | 2.8 | — | Continues treatment |
| | | response | | | | per protocol |
| | P8 | Partial | 6.7 | 2.8 | — | Continues treatment |
| | | response | | | | per protocol |
| | P9 | Partial | 5.3 | 2.8 | — | Continues treatment |
| | | response | | | | per protocol |

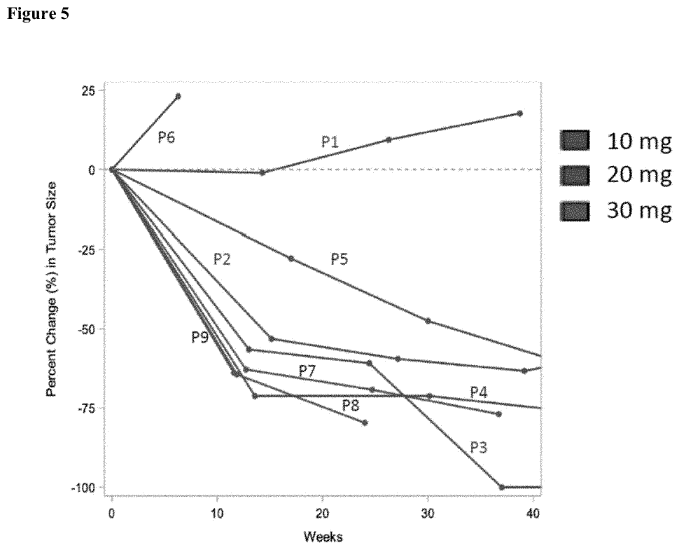

FIG. 5 shows a spider plot for tumor response for all the nine patients on the study (P1-P9). In dose level 1, two patients experienced tumor response, and one patient had stable disease (SD) as best response. The patient with SD (P1) had a mixed response (increase in size of axillary lymph node and decrease in size of subcutaneous lesions), ultimately coming off study to receive radiation to the growing axillary lymph node and progressing per imRECIST. The patient continues to remain on combination of pembrolizumab and propranolol off study (total follow up of 24.2 months).

In dose level 2, two patients had a PR, and one patient came off study due to the development of rapid disease progression. Both patients with PR discontinued combination therapy due to toxicity. One patient has maintained PR (total follow up of 18.9 months), whereas the other patient had PD and underwent metastasectomy for the residual metastatic lesion. In dose level 3, all three patients have experienced PR and continue on study treatment.

No DLTs were observed on the study. Observed frequency of adverse events were not higher than expected with pembrolizumab monotherapy alone. Two patients developed rare toxicities: HLH and ototoxicity. One patient developed HLH after 3 cycles of pembrolizumab, during admission for necrotizing fasciitis from methicillin-resistant Staphylococcus aureus (MRSA). Although rare, cases of HLH from pembrolizumab monotherapy have been reported in literature. Staphylococcus aureus is a known risk factor for secondary HLH and could have been a contributing cause. HLH resolved with steroids, without requirement of additional immunosuppression. No further episodes of HLH relapse have been observed until last follow up. Another patient developed grade 2 ototoxicity (hearing loss/labyrinthitis) after 2 cycles of pembrolizumab, which was treated with intratympanic and oral steroids. This patient had no evidence of leptomeningeal disease but still has residual hearing loss. Autoimmune hearing loss is also rare, but has been reported as a toxicity of pembrolizumab monotherapy. The mechanism of these rare irAE remains unclear. β-adrenergic receptors present in the inner ear epithelium play an important role in ion transportation. Some β-blockers have been implicated in hearing loss. Additionally, in rats, anti-PD1 have shown to be directly toxic to hair cell and organ of Corti. The inner ear is rich in melanocytes. Vogt-Koyanagi-Harada disease, an autoimmune disease targeting melanocytes frequently involves the inner ear. Ototoxicity in the described patient could also be due to a cross-reactive autoimmune response of the patient's T-cells to melanocytes in the inner ear.

In this Example, responses were observed at all 3 dose levels of propranolol with an objective response in 7/9 patients (78%). Five out of 7 patients continue to demonstrate maintenance of tumor response. After a median follow up of 15.6 months, 2/9 (22%) patients have gone on to receive second-line systemic therapy.

Chemokine and Cytokine Analysis

Intra-dose comparison: In dose level 1, 3-week (p=0.002), 6-week (p=0.008), 9-week (p=0.023) and 12-week (p=0.018) levels of IP-10 were significantly higher than at baseline. Immunosuppressive chemokine, eotaxin showed significant time effect in dose level 2 (p=0.04) with a large increase in expression around 12 weeks compared to baseline (2/3 patients). Additionally, there was a significant decrease at 12 weeks of immunostimulatory cytokine TNFβ compared to baseline in dose level 2 (p=0.003) (1/3 patients).

Inter-dose comparison: There was a decrease in expression of IL-12p70 in dose level 3 compared to dose levels 1 (p=0.007) and 2 (p=0.012) at week 6).

Responder vs. non-responder: At week 3, compared to baseline, IFN-γ was increased in dose level 1 and 2/3 patients in dose level 3 (all these were responders) and decreased in both non-responders. Interestingly, IL-6 decreased in 5/6 responders (value was not available for one responder) and increased in 1 non-responder (decreased in non-responder with mixed response; P1). An increase in IP-10 was observed among all patients.

Flow Cytometry

Intra-dose comparison: At all dose levels, there was a trend towards an initial increase in CD8⁺ T-cells/total CD45⁺ cells (CD8⁺T-cell %) until week 3. There was also a trend of early increase in the m-MDSC % and T-reg % at all dose levels. Interestingly, in dose level 1, 30-week m-MDSC % and PMN-MDSC % were significantly higher (p<0.01; p<0.01 respectively), whereas 30-week ratio of CD8⁺/PMN-MDSC was lower (p=0.01) when compared to baseline. A trend in early decrease in PMN-MDSC % were seen in the initial 3 weeks in dose levels 2 and 3.

Inter-dose comparison: Relative to baseline, significant decrease in PMN-MDSC % at 3 weeks was observed for dose level 2 compared to dose levels 1 (p=0.004) and 3 (p=0.007). Relative to baseline, highest increase in CD8⁺ T-cell % and ratio of CD8⁺T-cell/Treg was seen in dose level 3, although not statistically significant.

Responder vs. non-responder: There was significant heterogeneity among responders at different dose levels. Interestingly, at week 3, the ratio of CD8⁺T-cell/m-MDSC increased compared to baseline in 3/3 responders in dose level 3 and decreased/did not change in non-responders. An increase in CD8⁺ T-cell % compared to baseline was seen in 3/3 responders in dose level 3 vs. decrease in non-responder (increase in non-responder with mixed response; P1). An increase in Treg % was seen in all patients.

Tissue Biomarkers

Multispectral staining revealed marked variability in the composition and the distribution of immune infiltrate at the baseline. Non-responders (P1 and P6) had a lower number of CD8⁺ cytotoxic T cells, however three of the patients who responded to the therapy had comparable levels of CD8⁺ cytotoxic T cells, indicating that in a subset of patients, anti-tumor immune response can be activated independently of the baseline level. Number of FOXP3⁺ Tregs did not corelate with the response; in fact, several patients with disease control had higher baseline levels of Tregs, likely reflecting more brisk T cell infiltrate. The number of m-MDSC was highly variable among responders and non-responders. The patient with rapid disease progression had the highest number of PMN-MDSC (P6). Non-responders exhibited high expression of PD-L1 on m-MDSC or PMN-MDSCs relative to patients who benefited from the therapy. PD-L1+ melanoma cells varied from 0.34%-29.34% and did correlate with the response.

One of the non-responders (P6), had a clear cell morphology with minimal immune infiltration. Interestingly, this patient also had <1% PDL1+ melanoma cells, a higher infiltration of PMN-MDSC and a lower CD8⁺T-cell infiltration compared to other patients.

The described doses of propranolol show that the combination of pembrolizumab with propranolol is safe and does not result in an increase in toxicities compared to pembrolizumab monotherapy alone. The data also show the described combination does not compromise the efficacy of pembrolizumab monotherapy. Rather, based on the data in this Example, the present disclosure demonstrates that the combination is more effective than pembrolizumabas a monotherapy.

Although the present disclosure has been described using specific embodiments and examples, routine modifications will be apparent to those skilled in the art and such modifications are intended to be within the scope of the disclosure and the claims.

What is claimed is:

1. A method for therapy of cancer in a human individual in need thereof comprising administering to the human individual a β-blocker and an immune checkpoint inhibitor.

2. The method of claim 1, wherein the immune checkpoint inhibitor is pembrolizumab.

3. The method of claim 1, wherein the β-blocker is propranolol.

4. The method of claim 1, wherein the cancer comprises a melanoma tumor.

5. The method of claim 1, wherein the immune checkpoint inhibitor is the pembrolizumab and the β-blocker is the propranolol.

6. The method of claim 5, wherein the cancer comprises the melanoma tumor.

7. The method of claim 5, wherein the propranolol is administered in an amount that is at least 10 mg.

8. The method of claim 7, wherein the propranolol is administered in an amount that is 10 mg-30 mg.

9. The method of claim 8, wherein the propranolol is administered twice daily (BID).

10. The method of claim 9, wherein the propranolol is administered twice daily (BID) in an amount of 30 mg.

11. The method of claim 10, wherein the propranolol is administered twice daily (BID) in an amount of 30 mg and the pembrolizumab is administered once every three weeks.

12. The method of claim 11, wherein the pembrolizumab is administered in intravenously in an amount of 200 mg.

13. The method of claim 12, wherein the pembrolizumab is administered once every three weeks.

14. A method for therapy of melanoma in a human individual, the method comprising administering to the human individual a combination of propranolol and pembrolizumab.

15. The method of claim 14, wherein the propranolol is administered twice daily (BID) in an amount of 30 mg and the pembrolizumab is administered once every three weeks intravenously in an amount of 200 mg.
